(19) United States
(12) Patent Application Publication
(10) Pub. No.: US 2005/0266075 A1
(43) Pub. Date: Dec. 1, 2005

(54) Omeprazole dosage form

(75) Inventor: Chafic Chebli, Pierrefonds (CA)

Correspondence Address: Ronald S. Kosie;BCF LLP, 25th Floor, 1100 Rene-Levesque Boulevard West, Montreal, QC, H3B 5C9, CA

(73) Assignee: PHARMASCIENCE INC.

(21) Appl. No.: 11/139,592

(22) Filed: May 31, 2005

(30) Foreign Application Priority Data
Jun. 1, 2004 (CA) 2,469,427

(51) Int. Cl. A61K 31/4439; A61K 9/20

(52) U.S. Cl. 424/464; 514/338

(57) ABSTRACT

The present relates to a stable pharmaceutical composition comprising as an active component thereof one or more known 2-[(2-pyridyl)]-methylsulphinyl]benzimidazole derivatives

The present relates to a stable pharmaceutical composition comprising as an active component thereof one or more known 2-[(2-pyridyl)]-methylsulphinyl]benzimidazole derivatives (hereinafter sometimes simply referred to as “a gastric acid secretion inhibitor benzimidazole”, “gastric acid secretion inhibitor benzimidazoles”, “(a) proton pump inhibitor benzimidazole(s)”) as well as pharmaceutically acceptable salts thereof, pharmaceutically acceptable isomers thereof and pharmaceutically acceptable hydrates thereof. Thus, it is known that acid inhibitor benzimidazoles (or a pharmaceutically acceptable salt, isomers and hydrates thereof) may be useful as a gastric acid secretion inhibitor or proton pump inhibitor.

In general, an active benzimidazole component(s) may, for example, be a compound as described in EP patent 0 005129, or in Canadian patent no. 1264751. The entire contents of each of EP patent 0 005129 and Canadian patent no. 1264751 is incorporated herein by reference; similarly, the entire contents of all of the other patents and patent applications mentioned herein are also incorporated herein by reference.

However, the present invention will be described in relation to the exploitation of a group of particular known gastric acid secretion inhibitor benzimidazoles (or proton pump inhibitors), such as, for example, compounds known as omeprazole, lansoprazole, timoprazole, pariprazole, pantoprazole, etc as well as the pharmaceutically acceptable salts, isomers and hydrates thereof.

Particular acid secretion inhibitor benzimidazole salts (i.e. omeprazole salts) are, for example, described in Canadian patent no. 1264751; the structure of omeprazole is shown in this Canadian patent as follows (i.e. as 5-methoxy [[4-methoxy-3,5-dimethyl-2-pyridyl]-sulfinyl benzimidazole:

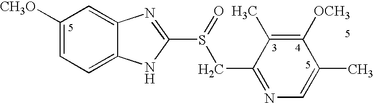

It is known that acid inhibitor benzimidazoles (as well as salts, etc., thereof) generally have poor stability in an acid medium.

It is known, for example, that an acid inhibitor benzimidazole such as, for example, omeprazole, is acid sensitive; i.e. in contact with an acidic reacting media it may degrade/transform to the point wherein it may lose its desired gastric acid suppressing activity. The degradation may be catalyzed by acidic reacting compounds. It is, for example, known to stabilize proton pump inhibitor(s) by associating them with alkaline reacting compounds, i.e. form mixtures thereof comprising alkaline reacting compounds.

With respect to the stability properties of the proton pump inhibitors mentioned above, it is, thus, known that an oral dosage form containing, a proton pump inhibitor (e.g. omeprazole), should be protected from contact with the acidic gastric juice in order for it to reach the small intestine without undesired degradation/transformation.

In the following, specific reference will be made, by way of example, only to omeprazole and/or pantoprazole; the comments apply, however, to the other acid inhibitor benzimidazoles (including salts, isomers and/or hydrates thereof) having similar acid sensitivity.

To alleviate the acid sensitivity for oral dosage forms containing omeprazole, it is known to apply an enteric coating over an omeprazole containing core or form. The purpose of the enteric coating is to protect the omeprazole, during passage of the dosage form through the stomach, from exposure to the acidic conditions of the stomach. However, enteric coating(s) may themselves have an acidic nature or character, which over time may also lead to acidic degradation/transformation of the active element of the dosage form. Thus, if such enteric-coated dosage form is stored under ambient conditions for a long period of time, the active component (e.g. omeprazole) may degrade to the point of ineffectiveness before it is administered to a patient.

To counter the acidic nature of an enteric coating it is further known to directly apply a separate inert intermediate coating between the core and the enteric coating; the purpose of such intermediate coating being to isolate the core from the acidic nature of an enteric coating. Such a separating inert layer between the core material comprising the pharmaceutically active substance, (e.g. omeprazole) and the enteric coating system is for instance described in Canadian patents nos. 1292693, 1302891 and 2166483, as well as published Canadian patent applications nos. 2184842, 2231223, 2258918, 2290531 and 2290824. Canadian patent no. 2186037, for example, describes an intermediate layer formed in situ between specific ingredients comprised in the core and the outer enteric coating.

A known alternate approach is to prepare the dosage form or core such that the form or core may be provided with an alkaline material able to offset the acidic nature of an enteric coating. It is in particular known for example to incorporate (an effective amount of) a basic inorganic salt stabilising agent or component into a dosage form or core containing an acid inhibitor or pharmaceutically acceptable salt thereof.

Canadian patent application no. 2377605, for example, resolves the problem of stabilization in favour of providing a core (containing an acid inhibitor benzimidazole) with an enteric coating which comprises an alkaline compound or agent (i.e. the enteric coating is to have a pH of at least 6.5 or higher). The reference, however, specifically teaches that the core is made by exploitation of a liquid based (i.e. water) or liquefaction type formulation step.

Canadian patent application no. 2382867 suggests that a gastric acid secretion (i.e. proton pump) inhibitor benzimidazole, namely 6-methoxy [[4-methoxy-3,5-dimethyl-2-pyridyl]-sulfinyl benzimidazole should be formulated as a dry blend in order to preserve the desired ratio of 6-methoxy[[4-methoxy-3,5-dimethyl-2-pyridyl]-sulfinyl benzimidazole to 5-methoxy[[4-methoxy-3,5-dimethyl-2-pyridyl]-sulfinyl benzimidazole. This document does not, however, teach a completely dry formulation process since the examples thereof show the use of a technique which includes some type of formulation component element or step which exploits a liquid or liquid state.

A more detailed description of various known types of systems for dealing with the problems associated with the protection during oral delivery of acid inhibitor gastric acid secretion (i.e. proton pump) inhibitor benzimidazole may be gleaned from the following: Canadian patent nos. 1292693, 1302891, 1338377, 2046364, 213762, 2166483, 2166794, 2170647 and 2284470, as well as published Canadian patent application nos. 2184842, 2186037, 2214033, 2231223, 2251430, 2258918, 2290531, 2290824, 2290893, 2310165, 2315261, 2319015, 2342209, 2346988, 2369951, 2383306, 2392353 and 2393483. Please also see U.S. Pat. No. 6,605,303.

It would be advantageous to be able to have relatively stable formulations comprising known gastric acid secretion inhibitor benzimidazoles such as for example omeprazole (i.e. known as a 5-methoxy-benzimidazole derivative), esomeprazole, lansoprazole, timoprazole, pariprazole, and/or pantoprazole, as well as pharmaceutically acceptable salts, isomers, and/or hydrates thereof, isomers include (e.g. optical isomers), enantiomers, racemates, etc. of these compounds.

It would be advantageous to be able to attenuate shelf life problems associated with the exploitation of an enteric coating without the need for an intermediate acid protection coating between the core and the enteric outer coating, i.e. to enhance storage stability. It would also be advantageous to be able to attenuate the use of a basic or alkaline (stabilizing) agent in a dosage form or core and if so desired or necessary to avoid the use of a basic or alkaline (stabilizing) agent altogether whether in the core or enteric coating.

### STATEMENT OF INVENTION

It has been surprisingly discovered that it is possible to provide a pharmaceutical solid unit dosage form (e.g. core) for oral administration comprising a gastric acid secretion (i.e. proton pump) inhibitor benzimidazole (i.e. at least one gastric acid secretion (i.e. proton pump) inhibitor benzimidazole as mentioned herein) and avoid the use of an intermediate acid protecting layer as well as, if so desired or necessary, also to diminish the use and/or even obviate the presence of alkaline material(s) in the dosage form or core or enteric coating.

Thus, the present invention in a general aspect provides a pharmaceutical solid unit dosage form (e.g. core) for oral administration comprising a gastric acid secretion (i.e. proton pump) inhibitor benzimidazole (i.e. at least one gastric acid secretion (i.e. proton pump) inhibitor benzimidazole), characterized in that
said solid unit dosage form is in a form prepared by direct compression of a dry powder prepared mixture comprising (e.g. consisting of) a gastric acid secretion (i.e. proton pump) inhibitor benzimidazole and a delivery vehicle.

The present invention in a further aspect provides a pharmaceutical solid unit dosage form (e.g. core) for oral administration comprising a gastric acid secretion (i.e. proton pump) inhibitor benzimidazole (i.e. at least one gastric acid secretion (i.e. proton pump) inhibitor benzimidazole), characterized in that
said solid unit dosage form is in a form prepared by direct compression of a dry powder prepared mixture comprising (e.g. consisting of) a gastric acid secretion (i.e. proton pump) inhibitor benzimidazole and a delivery vehicle,
said gastric acid secretion (i.e. proton pump) inhibitor benzimidazole being selected from the group consisting of 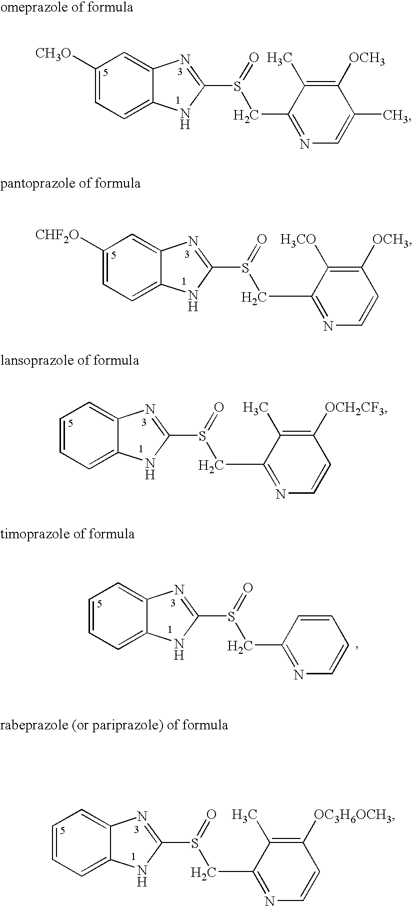
pharmaceutically acceptable salts, isomers and hydrates thereof,
and mixtures thereof and
said delivery vehicle comprising one or more members of the group consisting of pharmaceutically acceptable carriers, diluents and excipients.

In accordance with the present invention the dry prepared mixture may, as desired or necessary comprise an alkaline component (i.e. a basic or alkaline (stabilizing) agent(s)). However, in accordance with an advantageous aspect of the present invention, the dry prepared mixture may be free or at least essentially free of any alkaline component (i.e. at least essentially free of any basic or alkaline (stabilizing) agent); i.e. at least essentially free of any alkaline component as used, for example, in the dosage form or core described in Canadian Patent no. 2186037.

Thus, the present invention in another aspect provides a (dry formulated) pharmaceutical solid unit dosage form for oral administration comprising a gastric acid secretion (i.e. proton pump) inhibitor benzimidazole, characterized in that
said solid unit dosage form is in a form prepared by direct compression of a dry powder prepared mixture comprising a gastric acid secretion (i.e. proton pump) inhibitor benzimidazole and a delivery vehicle, and
wherein the dry prepared mixture is at least essentially free of any alkaline component (i.e. any basic or alkaline (stabilizing) agent).

The present invention in a further aspect provides a (dry formulated) pharmaceutical solid unit dosage form for oral administration comprising a gastric acid secretion (i.e. proton pump) inhibitor benzimidazole, characterized in that
said solid unit dosage form is in a form prepared by direct compression of a dry powder prepared mixture comprising a gastric acid secretion (i.e. proton pump) inhibitor benzimidazole and a delivery vehicle,
said gastric acid secretion (i.e. proton pump) inhibitor benzimidazole being selected from the group consisting of 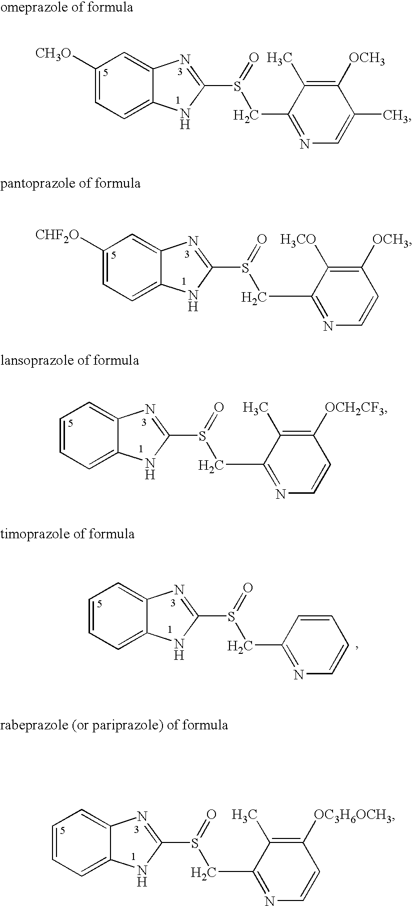
pharmaceutically acceptable salts, isomers and hydrates thereof
and mixtures thereof
said delivery vehicle comprising one or more members of the group consisting of pharmaceutically acceptable carriers, diluents and excipients and
wherein the dry prepared mixture is at least essentially free of any alkaline component (i.e. any basic or alkaline (stabilizing) agent).

Besides the active ingredient, the solid pharmaceutical unit dosage forms may (as discussed herein) include various conventional carriers, diluents and excipients such as fillers, disintegrants, binders, lubricants, surfactants, etc., and optionally colorants, and sweeteners.

In accordance with another aspect the present invention provides a process for the manufacture of a pharmaceutical solid unit dosage form for oral administration comprising a gastric acid secretion (i.e. proton pump) inhibitor benzimidazole, characterized in that said process comprises a solid unit dosage form formation step wherein said dosage form is prepared by direct compression of a dry powder prepared mixture comprising a gastric acid secretion (i.e. proton pump) inhibitor benzimidazole and a delivery vehicle.

In accordance with the process aspect of the present invention, the present invention provides a process for the manufacture of a pharmaceutical solid unit dosage form for oral administration comprising a gastric acid secretion (i.e. proton pump) inhibitor benzimidazole, characterized in that said process comprises a solid unit dosage form formation step wherein said dosage form is prepared by direct compression of a dry powder prepared mixture comprising a gastric acid secretion (i.e. proton pump) inhibitor benzimidazole and a delivery vehicle
said gastric acid secretion (i.e. proton pump) inhibitor benzimidazole being selected from the group consisting of 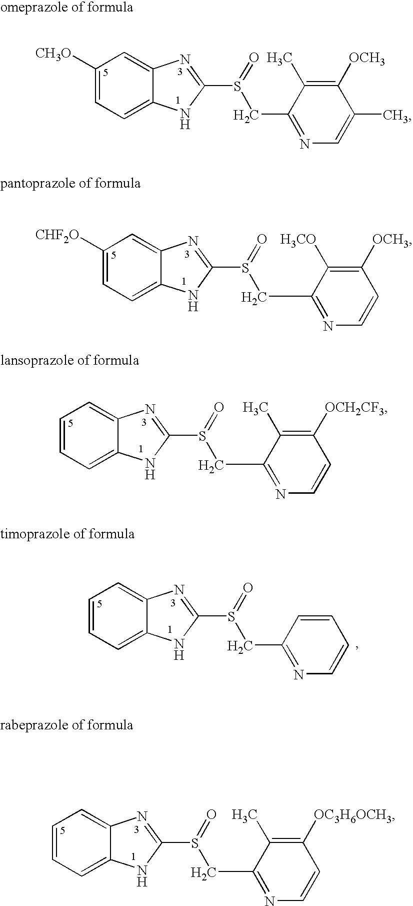
pharmaceutically acceptable salts, isomers and hydrates thereof,
and mixtures thereof,
said delivery vehicle comprising one or more members of the group consisting of pharmaceutically acceptable carriers, diluents and excipients.

In accordance with the present invention the dry prepared mixture for a process described herein may, as desired or necessary comprise an alkaline component (i.e. a basic or alkaline (stabilizing) agent(s)). However, in accordance with an advantageous aspect of the present invention, the dry prepared mixture for the process may be at least essentially free of any alkaline component (i.e. any basic or alkaline (stabilizing) agent).

In accordance with another aspect the present invention provides a (stable) pharmaceutical dosage formulation for oral administration which comprises (e.g. consists essentially of):
(a) a unit dosage core prepared by direct compression of a dry prepared mixture comprising a gastric acid secretion (i.e. proton pump) inhibitor benzimidazole and a delivery vehicle; and
(b) an enteric coating surrounding said unit dosage core, said enteric coating being applied directly to the unit dosage core without a separating coating between the enteric coating and said unit dosage core.

The gastric acid secretion (i.e. proton pump) inhibitor benzimidazole may be selected from the particular group of inhibitor benzimidazoles as described herein (e.g. omeprazole, lansoprazole, timoprazole, pariprazole, pantoprazole, etc as well as the pharmaceutically acceptable salts, isomers and hydrates thereof).

As mentioned above, in accordance with the present invention the dry prepared mixture may, as desired or necessary comprise an alkaline component (i.e. a basic or alkaline (stabilizing) agent(s)). However, in accordance with an advantageous aspect of the present invention, the dry prepared mixture may be free or at least essentially free of any alkaline component as described herein.

Furthemore, the enteric coating may, as desired or necessary comprise an alkaline component (i.e. a basic or alkaline (stabilizing) agent(s)). However, in accordance with an advantageous aspect of the present invention, the enteric coating may be at least essentially free of any alkaline component (i.e. may be obtained from the application onto the unit dosage form (e.g. core) of an enteric coating material which is at least essentially free of any alkaline component (i.e. at least essentially free of any basic or alkaline (stabilizing) agent)). Thus, the enteric coating material may have a pH of less than 6.5, i.e. be an enteric coating obtained by (or derived from) the application onto the outer surface of a dosage unit (or core) of an enteric coating forming composition able to form an enteric coating thereon (e.g. a solution material, a dispersion material, etc.), the enteric coating forming composition having a pH of less than 6.5 (e.g. a pH of less than 6.0, e.g. a pH of more than 4.0, e.g. a pH of less than 6.5 and higher than 4.0 (e.g. 6.5>pH>2.5), etc.).

Thus in accordance with the present invention a process described herein may further comprise an enteric coating application step wherein an enteric coating is applied directly on said dosage form so as to surround said dosage form without a separating layer between the enteric coating and said dosage form.

Thus, the present invention in a particular further aspect provides process for the manufacture of a (stable) pharmaceutical dosage formulation for oral administration comprising a gastric acid secretion (i.e. proton pump) inhibitor benzimidazole, characterized in that said process comprises (e.g. consists essentially of):
(a) a solid unit dosage form formation step wherein said dosage form is prepared by direct compression of a dry powder prepared mixture comprising a gastric acid secretion (i.e. proton pump) inhibitor benzimidazole and a delivery vehicle, wherein the dry prepared mixture is at least essentially free of any alkaline component (i.e. at least essentially free of any basic or alkaline (stabilizing) agent component); and
(b) an enteric coating application step wherein an enteric coating is applied directly on said dosage form so as to surround said dosage form without a separating layer between the enteric coating and said dosage form
said gastric acid secretion (i.e. proton pump) inhibitor benzimidazole being selected from the group consisting of 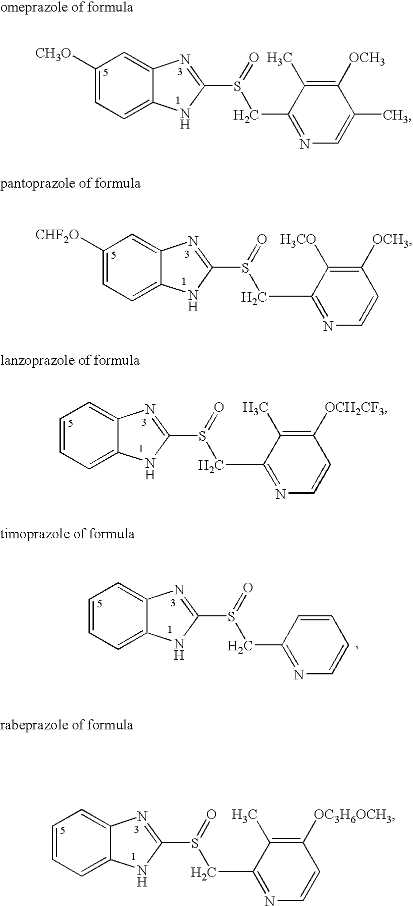
pharmaceutically acceptable salts, isomers and hydrates thereof,
and mixtures thereof,
said delivery vehicle comprising one or more members of the group consisting of pharmaceutically acceptable carriers, diluents and excipients.

It is to be understood herein, that if a “class”, “range”, “group of substances”, etc. is mentioned with respect to a particular characteristic (e.g., temperature, concentration, time, pressure, pH and the like) of the present invention, the present invention relates to and explicitly incorporates herein each and every specific member and combination of sub-classes, sub-ranges or sub-groups therein whatsoever. Thus, any specified class, range or group is to be understood as a shorthand way of referring to each and every member of a class, range or group individually as well as each and every possible sub-class, sub-range or sub-group encompassed therein; and similarly with respect to any sub-class, sub-ranges or sub-groups therein. Thus, for example,
with respect to the number of carbon atoms, the mention of the range of 1 to 6 carbon atoms is to be understood herein as incorporating each and every individual number of carbon atoms as well as sub-ranges such as, for example, 1 carbon atoms, 3 carbon atoms, 4 to 6 carbon atoms, etc.;
with respect to a pH range, it is to be understood as specifically incorporating herein each and every individual pH and pH range as well as sub-range (e.g. a ph of less than 6.5 specifically incorporates each and every individual pH and pH range as well as sub-range (e.g. a pH in the range of from less than 6.5 to 4.0, a ph of 5.3, a pH of 5.8, a ph of from 6.0 to 4.0, etc.));
with respect to a temperature range, it is to be understood as specifically incorporating herein each and every individual temperature and temperature range as well as sub-range;
with respect to time, such as a time of 1 minute or more is to be understood as specifically incorporating herein each and every individual time, as well as sub-range, above 1 minute, such as for example 1 minute, 3 to 15 minutes, 1 minute to 20 hours, 1 to 3 hours, 16 hours, 3 hours to 20 hours etc.;
and similarly with respect to any other parameters whatsoever, such as pressure, concentrations, elements, (carbon) atoms, etc. . . .

It is in particular to be understood herein that for any group or range, no matter how defined, a reference thereto is a shorthand way of mentioning and including herein each and every individual member described thereby as well as each and every possible class or sub-group or sub-class of members whether such class or sub-class is defined as positively including particular members, as excluding particular members or a combination thereof; for example an exclusionary definition for a formula may read as follows: “provided that when one of A and B is —X and the other is Y, —X may not be Z”.

It is also to be understood herein that “g” or “gm” is a reference to the gram weight unit and in relation to temperature “C”, or “C” is a reference to the Celsius temperature unit.

It is to be understood herein that the expression “dry powder prepared mixture” is a reference to a mixture which was prepared by intermingling of the components thereof, as is, i.e. in the absence of any supplementary aqueous or organic liquid solvent or any liquefaction (by heat) to facilitate intermingling of composition components. In other words the expression “dry prepared mixture” is a reference to a mixture which was prepared as a dry powder blend without recourse to any form of liquid phase to facilitate intermingling of the constituent components thereof; i.e. no liquid solvent or carrier including no melt carrier as described in Canadian patent application no. 2377605.

It is to be understood herein that the chemical name as well as the graphic formula of each of the particular benzimidazole compounds as shown and described herein includes, unless the contrary is stated or inferable, any and all isomers of any type whatsoever, including isomers such as optical and/or stereo isomers, such as, for example, enantiomers, racemates, diastereoisomers and the like.

As used herein, “direct compression” means that the solid unit dosage form is prepared by compression of a simple physical mixture of the active ingredient and delivery vehicle (e.g. excipients), without the active ingredient having been subjected to an intermediate wet or dry granulation process in order to embed it in a larger particle and improve its fluidity properties.

It is to be understood herein that a reference to an “enteric coating” is a reference to any (i.e. known) type of coating for protecting the benzimidazole gastric acid secretion (i.e. proton pump) inhibitor benzimidazole from degradation in the gastric acid medium after administration, but which coating does not inhibit the release of benzimidazole gastric acid secretion (i.e. proton pump) inhibitor benzimidazole into aqueous medium present in the small intestine, at pH values predominantly present in the small intestine.

It is to be understood herein that the expression “at least essentially free” as applied to the presence of an alkaline substance in a core and/or enteric coating is to be understood as qualifying a mixture as having no added alkaline substance or if present, alkaline substance is present in an amount which does not constitute or render it a stabilizing agent or an agent for in situ formation of an intermediate layer as described in the prior art; so as not for example to produce directly or indirectly a protective layer around the active ingredient particles. (i.e. as shown in Canadian patent no. 2186037).

It is to be understood herein that a gastric acid secretion (i.e. proton pump) inhibitor benzimidazole as described herein may be contained in or incorporated into a dosage form (e.g. core) in any desired or necessary therapeutically effective amount, i.e. in any known or desired amount.

In accordance with the present invention the gastric acid secretion (i.e. proton pump) inhibitor benzimidazole, may, for example, be selected from the group consisting of omeprazole, pharmaceutically acceptable salts, isomers and hydrates thereof and mixtures thereof.

In accordance with the present invention the gastric acid secretion (i.e. proton pump) inhibitor benzimidazole, may be selected from the group consisting of omeprazole and a magnesium salt of omeprazole.

In accordance with the present invention the gastric acid secretion (i.e. proton pump) inhibitor benzimidazole may comprise or be esomeprazole, an isomer of omeprazole, of formula

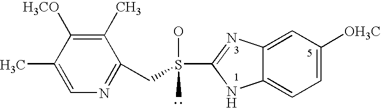

The present invention in a further aspect provides a pharmaceutical dosage formulation for oral administration which consists essentially of:
(a) a dosage core prepared by direct compression of a dry prepared mixture comprising (e.g. consisting of) a delivery vehicle and an gastric acid secretion (i.e. proton pump) inhibitor benzimidazole selected from the group thereof described herein; and
(b) an enteric coating surrounding said core, said enteric coating being applied directly to the core without a separating layer between the enteric coating and said core.

As mentioned above, a pharmaceutical dosage formulation, in accordance with the present invention may be further characterized in that the dry prepared mixture may if so desired or necessary be at least essentially free of any alkaline component (i.e. at least essentially free of any basic or alkaline (stabilizing) agent), i.e. the composition of a dry prepared mixture may be subject to such a proviso.

In accordance with the present invention gastric acid secretion (i.e. proton pump) inhibitor benzimidazole may be selected from the group consisting of omeprazole and pharmaceutically acceptable salts thereof (e.g. a magnesium salt of omeprazole).

In accordance with the present invention the delivery vehicle may comprise (e.g. consist of) a filler component (e.g. one or more fillers), a binding agent component (e.g. one or more binding agents), a solubilizing agent component (e.g. one or more solubilizing agents) and a surfactant component (e.g. one or more surfactants).

In accordance with the present invention the delivery vehicle may alternatively comprise (e.g. consist of) a filler component (e.g. one or more fillers), a binding agent component (e.g. one or more binding agents), a solubilizing agent component (e.g. one or more solubilizing agents), a surfactant component (e.g. one or more surfactants), a disintegrating agent component (e.g. one or more disintegrating agents) and a lubricant component (e.g. one or more lubricants).

Thus the present invention in a particular aspect a pharmaceutical dosage formulation for oral administration (as well as a process for the manufacture or preparation thereof) wherein said dry prepared mixture comprises (e.g. consists essentially of) a gastric acid secretion (i.e. proton pump) inhibitor benzimidazole component, a surfactant component, a filler component, a binding agent component and a solubilizing agent component; said dry prepared mixture comprising
an amount of said benzimidazole component sufficient to provide said benzimidazole component in an amount in the range of from 5 mg to 60 mg per dosage core,
an amount of said surfactant component sufficient to provide from 0.5 to 5.0 weight percent, based on the total weight of the dosage core, of said surfactant component per dosage core,
an amount of said filler component sufficient to provide from 5.0 to 85.0 weight percent based on the total weight of the core of said filler component per dosage core,
an amount of said binding agent component sufficient to provide from 1.0 to 20.0 weight percent based on the total weight of the core of said binding agent component per dosage core and
an amount of said solubilizing agent component sufficient to provide from 2.0 to 25 weight percent based on the total weight of the core of said solublizing agent component per dosage core.

In accordance with the present invention a gastric acid secretion (i.e. proton pump) inhibitor benzimidazole component may of course be a benzimidazole substance as described herein.

In accordance with the present invention there is also provided a pharmaceutical dosage formulation wherein said dry prepared mixture further comprises a disintegrating agent component, and a lubricant component, said dry prepared mixture comprising
an amount of said disintegrating agent component sufficient to provide from 0.5 to 8.0 weight percent based on the total weight of the core of said disintegrating agent component per dosage core and
an amount of said lubricant agent component sufficient to provide from 0.05 to 5.0 weight percent based on the total weight of the core of said lubricant agent component per dosage core.

In accordance with the present invention the enteric coating may take on any desired or necessary form. The enteric coating may for example be a methacrylic acid copolymer coating. In accordance with the present the enteric coating may, for example, be a sugar coating.

More generally an enteric coating (layer) may be an enteric film coating polymer, such as cellulose acetate phtalate, hydroxypropyl methylcellulose phtalate, polyvinyl acetate phtalate, carboxymethylcellulose, co-polymerized methacrylic/methacylic acid methyl esters such as for instance, compounds known under the trade name Eudragit L12.5 or Eudragit L 100 (Röhm Pharma), or similar compounds used to obtain enteric coatings. The enteric coating may also be applied using water-based polymer dispersions, e.g. Aquateric (FMC Corporation), Eudragit L100-55 (Röhm Pharma), Coating CE 5142 (BASF). The enteric coating may as mentioned in particular be of methacrylic acid copolymer (i.e. Acryl-eze, a brand name of Colorcon). An enteric coating may be a sugar coating ensuring the acid inhibitor benzimidazole protection in the gastric acid medium.

In accordance with the present invention an excipient may be a filler (i.e. at least one filler), a binding agent (i.e. at least one binding agent), a disintegrating agent (i.e. at least one disintegrating agent), a solubilizing agent (i.e. at least one solubilizing agent), and a lubricant (i.e. at least one lubricant), and/or a surfactant (i.e. at least one surfactant).

In accordance with the present invention a solid dosage form or core may, for example, comprise at least one (i.e. one or more) filler selected from the group consisting of, cellulose, microcrystalline cellulose, silicified microcrystalline cellulose, cellulose acetate, sugar, dextrate, dextrin, dextrose, ethyl cellulose, sorbitol, fructose, mannitol, fumaric acid, lactitol, lactose, maltose, sodium alginate, starch, pregelatinized starch, maize starch, sucrose, sugar spheres, talc, xylitol, tragacanth, trehalose, xylitol, polymethacrylates, glyceryl palmitostearate, hydrogenated vegetable oil, kaolin, maltodextrin, medium chain triglycerides. Most preferably the filler is Lactose.

In accordance with the present invention a solid dosage form or core may, for example, comprise at least one (i.e. one or more) binder selected from the group consisting of acacia, alginic acid, carbomers, carboxymethylcellulose sodium, carrageenan, cellulose acetate phtalate, chitosan, glucose, dextrose, dextrate, dextrin, ethyl cellulose, microcrystalline cellulose, sugar, glyceryl behenate, guar gum, hydroxyethylcellulose, hydroxymethylcellulose, hydroxypropylcellulose, hydroxypropylmethyl cellulose, hypromellose, methylcellulose, poloxamer, polydextrose, polyethylene oxide, polymethacrylates, povidone, stearic acid, zein. Most preferably the binder is hydroxymethylpropyl cellulose.

In accordance with the present invention a solid dosage form or core may, for example, comprise at least one (i.e. one or more) disintegrating agents selected from the group consisting of alginic acid, carboxymethylcellulose calcium, carboxymethylcellulose sodium, crospovidone, cellulose, chitosan, colloidal silicon dioxide, croscarmellose sodium, guar gum, hydroxypropyl cellulose, methylcellulose, microcrystalline cellulose, povidone, sodium alginates, sodium starch glycolate, starch. Most preferably the disintegrating agent is croscarmellose and/or sodium starch glycolate.

In accordance with the present invention a solid dosage form or core may, for example, comprise at least one (i.e. one or more) solubilizing agents selceted from the group consisting of, cyclodextrins, meglumine, poloxamer, polyethylene glycol (solid grades), povidone, stearic acids. Most preferably the solubilizing agent or surfactant is polyethylene glycol.

In accordance with the present invention a solid dosage form or core may, for example, comprise at least one (i.e. one or more) surfactants selected from the group consisting of sodium lauryl sulfate, butylparaben, ethylparaben, methylparaben, propylparaben, sorbic acid, Most preferably the surfactant is sodium lauryl sulphate.

In accordance with the present invention a solid dosage form or core may, for example, comprise at least one (i.e. one or more) lubricants selected from the group consisting of, hydroxyethyl cellulose, poloxamers, polyvinyl alcohol, talc, calcium stearate, glyceryl behenate, glyceryl palmitostearate, hydrogenated vegetable oil, magnesium lauryl sulfate, magnesium stearate, medium chain triglycerides, sodium lauryl sulfate, sodium stearyl fumarate, stearic acid, zinc stearate. Most preferably the lubricant is sodium stearyl fumarate.

In particular the filler may be Lactose; the binder may be hydroxymethylpropyl cellulose; the disintegrating agent may be selected from the group consisiting of croscarmellose, sodium starch glycolate and mixtures thereof; the solubilizing agent may be polyethylene glycol; the surfactant may be sodium lauryl sulphate; and the lubricant may be sodium stearyl fumarate.

The invention will now be discussed in relation to the formation a composition in the form of a tablet.

Core or Dosage Form

An acid inhibitor benzimidazole substance may be simply physically mixed with inert, (for example advantageously water soluble), conventional pharmaceutical excipients, in conventional blenders. The intermingling is of course to be carried out in the absence of any supplementary aqueous or organic based solvent systems. If desired the intermingling may as well be carried out in the absence of any supplementary basic or alkaline agent; for the examples referred to below no supplementary basic or alkaline agent was used. The components are intermingled in sufficient amounts in order to obtain the necessary or desired concentration of acid inhibitor benzimidazole substance in the final mixture. For the example embodiments referred to below, the final mixture contained no alkaline reacting compounds for creating a basic pH micro-environment around the acid inhibitor benzimidazole substance for enhancing its stability as widely described in the prior art. In other words for the example embodiments mentioned below the Omeprazole core formulation contained no alkaline reacting compounds and yet surprisingly the Omeprazole formulation was stable.

The final powder mixture may then be processed into tablets or mini-tablets using conventional tablet Press machines, such as Colton 2216, B-tooling rotary tablet press machine.

Coating Layer

As mentioned above, after administration a tablet will travel through the gastro-intestinal tract, and if unprotected will be exposed to gastric acid medium, which causes degradation/discoloration of the acid inhibitor benzimidazole substance. Thus dry obtained dry mixed tablets or mini-tablets (i.e. cores) are treated so as to be provided with an enteric coating for protection against any direct contact with the acid gastric medium of the gastro-intestinal tract (i.e. GIT). If desired, a single enteric layer may be directly applied to the core by conventional coating procedures in a suitable coating pan or in fluidized bed apparatus using water and/or conventional organic solvents for the coating solution. Alternatively, if so desired two or more of such enteric layers may be laid down one after the other so as to define the enteric coating. In any case however no intermediate inert layer is laid down between the core and the enteric coating.

A non-functional conventional color coat on top of the said enteric coating may be optionally be added for marketing purposes, if so desired.

Final Dosage Form

The final dosage form is either coated tablets or mini-tablets. The mini-tablets may be filled in hard capsule shells or sachets ensuring the stability of the benzimidazole in gastric medium. Advantageously, for the long-term stability during shelf-life, the final packaging may contain a desiccant Censuring a low water content.

Proposed range of tablet weight and percentage for each excipient
of the total weight of the tablet¹ (DR = delayed release)

| | Actual | Range of |
|---|---|---|
| | weight of | Weight of |
| Formulation | tablet | tablet |
| Example 1 to 4: | | |
| Omeprazole DR tablets 20 mg | 175 mg | 20-500 mg |
| Omeprazole DR tablets 10 mg | 87.5 mg² | 10-300 mg |
| Examples 5-6: | | |
| Pantoprazole DR tablets 40 mg | 200 mg | 20-500 mg |
| Pantoprazole DR tablets 20 mg | 100 mg | 10-300 mg |

¹Excipients' percentages include those of Pantoprazole formulation examples
²If both strengths are proportional

| | Range of |
|---|---|
| Ingredients used in Formulation examples of | percentage of the |
| Omeprazole and/or Pantoprazole | total weight of the tablet |
| Lactose (Filler) | 5-85% |
| Microcrystalline Cellulose (Filler) | 5-85% |
| Polyethylene glycol (Solubilizing agent) | 2-25% |
| Glyceryl monostearate (Solubilizing agent) | 2-25% |
| Croscarmellose Sodium (Disintegrating agent) | 0.5-8% |
| Crospovidone (Disintegrating agent) | 0.5-8% |
| Sodium Starch Glycolate (Disintegrating agent) | 0.5-8% |
| Hydroxypropylmethyl cellulose (Binding agent) | 1-15% |
| Povidone (Binding agent) | 2-20% |
| Sodium Stearyl Fumarate (Lubricating agent) | 0.05-5% |
| Magnesium stearate (Lubricant) | 0.05-5% |
| Colloidal silicon dioxide 200 (Glidant) | 0.01-5% |
| Sodium Lauryl Sulfate (Surfactant) | 0.5-5% |

### EXAMPLES

The following Examples illustrate example embodiments of the present invention. In each case the acid inhibitor benzimidazole is Omeprazole base or Omeprazole magnesium salt. The other core components were as set forth below. The process to manufacture the core, comprised first thoroughly blending the acid inhibitor benzimidazole directly with the solubilizing agent and/or the surfactant in order to further enhance the acid inhibitor benzimidazole dissolution and consequently its absorption in vivo. The other conventional pharmaceutical excipients were then added on the so obtained benzimidazole pre-mix and blended to yield the final mixture. The final mixture was also prepared in the absence of any aqueous or organic solvent-based system. Finally, the dry blended mixture is directly compressed to yield the desired cores.

### Example 1

This example of the composition of the present invention was prepared as follows. The core containing the benzimidazole was prepared by direct compression of all excipients into tablets. The core is then coated with only one layer, namely an enteric coating layer applied directly on the said core.

### Example 1

### Formulation of the Said Core

| Ingredients | Quantity per tablet | % per tablet weight |
|---|---|---|
| Omeprazole Magnesium | 20.5 mg | 11.76 |
| Pharmatose 50 M | 87.0 mg | 49.74 |
| PEG 8000 | 38.5 mg | 22.00 |
| Croscarmellose Sodium | 3.5 mg | 2.00 |
| Sodium Starch Glycolate | 8.8 mg | 5.00 |
| Hydroxypropylmethyl | 8.8 mg | 5.00 |
| cellulose | | |
| Sodium Stearyl Fumarate | 4.4 mg | 2.50 |
| Sodium Lauryl Sulfate | 3.5 mg | 2.00 |

For the preparation of the core, Omeprazole Magnesium was mixed thoroughly with PEG 8000 (solubilizing agent-polyethylene glycol from Dow Chemical) and Sodium Lauryl Sulfate (surfactant) and the mixture was then sieved so as to obtain the first premix.

Pharmatose 50 M (a filler-lactose from Borculodomo Ingredients, Netherlands) and Croscarmellose Sodium (a disintegrant—crosslinked carboxymethylcellulose) were directly added to the previous mixture and mixed thoroughly therewith as to provide the second premix

Sodium Starch Glycolate (a disintegrant—sodium carboxymethyl starch) and Hydroxypropylmethyl cellulose (a binder) were directly mixed aside, then sieved and added to the second premix above as to obtain the third premix.

Sodium Stearyl Fumarate (a lubricant—2-butenedioic acid, monooctadecyl ester, sodium salt) was sieved, added directly to the third premix above and mixed thoroughly therewith as to obtain a final dry blend. The blend was compressed into tablets using a conventional tablet press such as Colton 2216 Rotary tablet press; each tablet contained the equivalent of 20 mg of Omeprazole.

Tablets were then transferred into a conventional coating pan and coated with only one layer, namely an enteric coating layer using any conventional tablet coater machine such as Labcoat, OHARA, California USA, applied directly on the said core, prepared in the following manner.

### Example 1

### Enteric Coating Layer

| Ingredients | Quantity per tablet |
|---|---|
| Acryl-Eze | 21.0 mg |
| Antifoam emulsion | 0.70 mg |

First, the antifoam emulsion (a silicone antifoam emulsion—DOW CORNING) was dissolved in water to form an aqueous solution. An enteric coating system (Acryl-Eze, brand name of Colorcon—Westpoint, USA) was then added slowly into this solution for a final concentration of about 15% of weight per total weight of the solution. The coating solution was stirred constantly while sprayed (EUROSTAR mixer) onto the tablets with an incoming air temperature of 40° C.

It was determined, after storage in high density polyethylene bottles, with no desiccant, under ambient conditions (i.e. at 20-25° C. and 45-70% RH(RH=relative humidity)) that the tablets had acceptable degradation stability after six months.

### Example 2

The core containing the active material was prepared by direct compression of all excipients into tablets. The core is then coated with only one layer, namely enteric coating layer using Acryl-Eze.

### Example 2

### Formulation of the Core

| | | % per |
|---|---|---|
| Ingredients | Quantity per tablet | tablet weight |
| Omeprazole | 20.0 mg | 11.35 |
| Pharmatose 50 M | 53.3 mg | 30.65 |
| Glyceryl monostearate | 26.3 mg | 15.00 |
| Crospovidone | 8.8 mg | 5.00 |
| Hydroxypropylmethyl cellulose | 8.8 mg | 5.00 |
| Microcrystalline cellulose | 52.5 mg | 30.00 |
| Sodium Stearyl Fumarate | 5.3 mg | 3.00 |

For the preparation of the core, Omeprazole was mixed thoroughly with Pharmatose 50M and glyceryl monostearate (a solubilizing agent—octadecanoic acid, monoester with 1,2,3-propanetriol) and the mixture was then sieved as to obtain the first premix.

Crospovidone (a disintegrant —1-ethenyl-2 pyrrolidinone homopolymer), Vivapur 12 (a filler—microcristalline cellulose from J. Rettenmaier & Sohn, Germany) and hydroxypropylmethyl cellulose (a binder) were directly mixed aside, then sieved and the obtained sieved mixture was added to the first premix above as to obtain the second premix.

Sodium Stearyl Fumarate was sieved, added to the second premix above and mixed thoroughly as to obtain the final dry blend.

The obtained dry blend was compressed into tablets using a conventional tablet press such as Colton 2216 Rotary tablet press; each tablet contained the equivalent of 20 mg of Omeprazole.

Tablets were transferred into a conventional coating pan and coated with only one layer, namely an enteric coating layer using any conventional tablet coater machine such as Labcoat, OHARA, applied directly on the said core, prepared in the following manner.

### Example 2

### Enteric Coating Layer

| Ingredients | Quantity per tablet |
|---|---|
| Acryl-Eze | 17.5 mg |
| Antifoam emulsion | 0.60 mg |

First, the antifoam emulsion (20% active silicone antifoam emulsion—DOW COMING) was dissolved in water to form an aqueous solution. An enteric coating system (Acryl-Eze) was then added slowly into this solution for a final concentration of about 15% of weight per final weight of the solution. The coating solution was stirred constantly while sprayed onto the tablets with an incoming air temperature of 40° C.

It was determined, after storage in high density polyethylene bottles, with no desiccant, under ambient conditions (i.e. at 20-25° C. and 45-70% RH(RH=relative humidity)) that the tablets had acceptable degradation stability after ten months.

### Example 3

This example of the composition of the present invention was prepared as follows. The core containing the active material was prepared by direct compression of all excipients into tablets.

### Example 3

### Formulation of the Core

| | | % per |
|---|---|---|
| Ingredients | Quantity per tablet | tablet weight |
| Omeprazole Magnesium | 20.5 mg | 11.76 |
| Pharmatose 50 M | 110.7 mg | 63.24 |
| PEG 8000 | 17.5 mg | 10.00 |
| Croscarmellose Sodium | 3.5 mg | 2.00 |
| Sodium Starch Glycolate | 8.8 mg | 5.00 |
| Hydroxypropylmethyl cellulose | 8.8 mg | 5.00 |
| Sodium Stearyl Fumarate | 4.4 mg | 2.50 |
| Sodium Lauryl Sulfate | 0.9 mg | 0.50 |

For the preparation of the core, Omeprazole Magnesium was directly mixed thoroughly with PEG 8000 and Sodium Lauryl Sulfate and the mixture was then sieved so as to obtain the first premix.

Pharmatose 50 M and Croscarmellose Sodium were directly added to the first premix and mixed thoroughly therewith so as to obtain the second premix.

Sodium Starch Glycolate and Hydroxypropylmethyl cellulose were directly mixed aside then sieved and the sieved mixture added to the second premix above so as to obtain the third premix

Sodium Stearyl Fumarate was sieved, the sieved product was then added to the third premix above and mixed thoroughly so as to obtain the final dry blend.

The obtained dry blend was compressed into tablets using a conventional tablet press such as Colton 2216 Rotary tablet press; each tablet contained the equivalent of 20 mg of Omeprazole.

It was determined, after storage in high density polyethylene bottles, with no desiccant, under ambient conditions (i.e. at 20-25° C. and 45-70% RH(RH=relative humidity)) that the tablets had acceptable degradation stability.

### Example 4

### Retained Formulation: Passed Pilot Bioequivalence Study

This example of the composition of the present invention was prepared as follows. The core containing the benzimidazole was prepared by direct compression of all excipients into tablets. The core is then coated with only one layer, namely an enteric coating layer applied directly on the said core.

### Example 4

### Formulation of the Said Core

| Ingredients | Quantity per tablet | % per tablet weight |
|---|---|---|
| Omeprazole Magnesium | 20.6 mg | 11.80 |
| PEG 8000 | 17.6. mg | 10.00 |
| Sodium Lauryl Sulfate | 0.9 mg | 0.50 |
| Croscarmellose Sodium | 3.5 mg | 2.00 |
| Povidone S-630 | 21.0 mg | 12.00 |
| Pharmatose 50 M | 54.6 mg | 31.20 |
| MCC PH 112 | 43.8 mg | 25.00 |
| Sodium Starch Glycolate | 8.8 mg | 5.00 |
| Sodium Stearyl Fumarate | 4.4 mg | 2.50 |

For the preparation of the core, Omeprazole Magnesium was mixed thoroughly with PEG 8000 and Sodium Lauryl Sulfate and the mixture was then sieved so as to obtain the first premix.

Pharmatose 50 M, Croscarmellose Sodium, MCC PH 112 (a filler—microcrystalline cellulose), Sodium Starch Glycolate and Povidone S-630 (a binder-1-ethenyl-2 pyrrolidinone homopolymer) were mixed thoroughly, sieved and directly added to the first premix as to obtain the second premix.

Sodium Stearyl Fumarate was sieved, added directly to the second premix above and mixed thoroughly therewith as to obtain a final dry blend. The blend was compressed into tablets using a conventional tablet press such as Colton 2216 Rotary tablet press; each tablet contained the equivalent of 20 mg of Omeprazole.

Tablets were then transferred into a conventional coating pan and coated with only one layer, namely an enteric coating layer using any conventional tablet coater machine such as Labcoat, OHARA, applied directly on the said core, prepared in the following manner.

### Example 4

### Enteric Coating Layer

| Ingredients | Quantity per tablet |
|---|---|
| Acryl-Eze | 21.0 mg |
| Antifoam emulsion | 0.70 mg |

First, the antifoam emulsion (silicone antifoam emulsion—DOW COMING) was dissolved in water to form an aqueous solution. An enteric coating system (Acryl-Eze, brand name of Colorcon—Westpoint, USA) was then added slowly into this solution for a final concentration of about 15% of weight per total weight of the solution. The coating solution was stirred constantly while sprayed (EUROSTAR mixer) onto the tablets with an incoming air temperature of 40° C.

It was determined, after storage in high density polyethylene bottles, with no desiccant, under ambient-conditions (i.e. at 20-25° C. and 45-70% RH(RH=relative humidity)) that the tablets had acceptable degradation stability after two months.

### Example 5

This example of the composition of the present invention was prepared as follows. The core containing the benzimidazole was prepared by direct compression of all excipients into tablets. The core is then coated with only one layer, namely an enteric coating layer applied directly on the said core.

### Example 5

### Formulation of the Said Core

| Ingredients | Quantity per tablet | % per tablet weight |
|---|---|---|
| Pantoprazole Sodium | 46.1 mg | 23.05 |
| Sesquihydrate | | |
| Lactose Fast Flow | 40.0 mg | 20.00 |
| Crospovidone | 10.0 mg | 5.00 |
| Vivapur 12 | 69.9 mg | 34.95 |
| PVP S-630 | 30.0 mg | 15.00 |
| Magnesium Stearate | 3.0 mg | 1.50 |
| Colloidal Silicon Dioxide | 1.0 mg | 0.50 |
| 200 | | |

For the preparation of the core, Pantoprazole sodium sesquihydrate was mixed thoroughly with Vivapur 12 (a filler—microcrystalline cellulose) and Crospovidone (a disintegrant —1-ethenyl-2 pyrrolidinone homopolymer) and the mixture was then sieved so as to obtain the first premix.

Lactose fast flow (a filler—lactose from Foremost Farms USA) and PVP S-630 (a binder—1-ethenyl-2 pyrrolidinone homopolymer) were sieved and directly added to the previous mixture and mixed thoroughly therewith as to provide the second premix.

Magnesium stearate (lubricant) and Colloidal Silicon Dioxide 200 (a glidant from Calmags, Denmark) were directly mixed aside, then sieved and added to the second premix above as to obtain the final dry blend. The blend was compressed into tablets using a conventional tablet press such as Colton 2216 Rotary tablet press; each tablet contained the equivalent of 40 mg of Pantoprazole.

Tablets were then transferred into a conventional coating pan and coated with only one layer, namely an enteric coating layer using any conventional tablet coater machine such as Labcoat, OHARA, applied directly on the said core, prepared in the following manner.

### Example 5

### Enteric Coating Layer

| Ingredients | Quantity per tablet |
|---|---|
| Acryl-Eze | 24.0 mg |
| Antifoam emulsion | 0.80 mg |

First, the antifoam emulsion (silicone antifoam emulsion—DOW COMING) was dissolved in water to form an aqueous solution. An enteric coating system (Acryl-Eze, brand name of Colorcon—Westpoint, USA) was then added slowly into this solution for a final concentration of about 15% of weight per total weight of the solution. The coating solution was stirred constantly while sprayed (EUROSTAR mixer) onto the tablets with an incoming air temperature of 40° C.

It was determined, after storage in high density polyethylene bottles, with no desiccant, under ambient conditions (i.e. at 20-25° C. and 45-70% RH(RH=relative humidity)) that the tablets had acceptable degradation stability after twelve months.

### Example 6

This example of the composition of the present invention was prepared as follows. The core containing the benzimidazole was prepared by direct compression of all excipients into tablets. The core is then coated with only one layer, namely an enteric coating layer applied directly on the said core.

### Example 6

### Formulation of the Said Core

| Ingredients | Quantity per tablet | % per tablet weight |
|---|---|---|
| Pantoprazole Sodium | 45.1 mg | 22.54 |
| Sesquihydrate | | |
| Pharmatose 50 M | 40.0 mg | 20.00 |
| Sodium Lauryl Sulfate | 2.0 mg | 1.00 |
| Sodium Starch Glycolate | 8.0 mg | 4.00 |
| Vivapur 12 | 59.3 mg | 29.67 |
| PEG 8000 | 10.0 mg | 5.00 |
| PVP S-630 | 26.0 mg | 13.00 |
| Croscarmellose Sodium | 4.0 mg | 2.00 |
| Magnesium Stearate | 3.0 mg | 1.50 |
| Colloidal Silicon Dioxide | 2.6 mg | 1.30 |
| 200 | | |

For the preparation of the core, Pantoprazole sodium sesquihydrate was mixed thoroughly with Vivapur 12 and Sodium Starch Glycolate and Sodium Lauryl Sulfate and the mixture was then sieved so as to obtain the first premix.

Pharmatose 50 M, PEG 8000 (solubilizing agent—polyethylene glycol), Croscarmellose Sodium and PVP S-630 were sieved and directly added to the previous mixture and mixed thoroughly therewith as to provide the second premix.

Magnesium stearate (lubricant) and Colloidal Silicon Dioxide (glidant) were directly mixed aside, then sieved and added to the second premix above as to obtain the final dry blend. The blend was compressed into tablets using a conventional tablet press such as Colton 2216 Rotary tablet press; each tablet contained the equivalent of 40 mg of Pantoprazole.

It was determined, after storage in high density polyethylene bottles, with no desiccant, under ambient conditions (i.e. at 20-25° C. and 45-70% RH(RH=relative humidity)) that the tablets had acceptable degradation stability.

### Example 7

### pH Determination of Formulations of Enteric Coating Forming Compositions

Equipment: Mettler Toledo MP 230 pH meter, Sartorius Balance, Cimarec Stirrer plate

Enteric Coating Material:

Acryl-Eze—colour pink (manufacturer product code Acryl-Eze pink 93014318),
Acryl-Eze—colour pink (manufacturer product code Acryl-Eze pink 93014474)
Acryl-Eze—colour yellow (manufacturer product code Acryl-Eze yellow 93012268),
   Method:

For each of the above coating materials, there was prepared a solution of 10%, 20% & 30% by weight of coating material per weight of solution using water as a solvent. The pH of each of the solutions as well as the pH of the water used was taken.

Results:

PH of water sampled on the same day: 8.745

TABLE I
ACRYL-EZE PINK 93014318 (Omeprazole DR tablets 20 mg)

| QUANTITY | TOTAL VOLUME | |
|---|---|---|
| OF PRODUCT | OF SOLUTION | |
| (g) | (ml) | PH (At 25° C.) |
| 10.0052 | 100 | 5.652 |
| 20.0105 | 100 | 5.467 |
| 30.02 | 100 | 5.422 |

TABLE II
ACRYL-EZE PINK 93014474 (Omeprazole DR tablets 10 mg)

| QUANTITY | TOTAL VOLUME | |
|---|---|---|
| OF PRODUCT | OF SOLUTION | |
| (g) | (ml) | PH (At 25° C.) |
| 10.0073 | 100 | 5.741 |
| 20.0023 | 100 | 5.467 |
| 30.06 | 100 | 5.373 |

TABLE III
ACRYL-EZE YELLOW 93012268 (Pantoprazole DR tablets 20 & 40 mg)

| QUANTITY | TOTAL VOLUME | |
|---|---|---|
| OF PRODUCT | OF SOLUTION | |
| (g) | (ml) | PH (At 25° C.) |
| 10.0012 | 100 | 5.721 |
| 20.0101 | 100 | 5.476 |
| 30.12 | 100 | 5.321 |

### CONCLUSION

As observed in table I, II & III, the pH of the coating layer forming composition does not exceed 5.8 for all solutions ranging from 10% until 30% of weight per weight of solution.

It is to be noted of course that other types of enteric coating forming compositions may have lower pH values.

1. A pharmaceutical solid unit dosage form for oral administration comprising a gastric acid secretion inhibitor benzimidazole, characterized in that
said solid unit dosage form is in a form prepared by direct compression of a dry prepared mixture comprising a gastric acid secretion inhibitor benzimidazole and a delivery vehicle,
said gastric acid secretion inhibitor benzimidazole being selected from the group consisting of 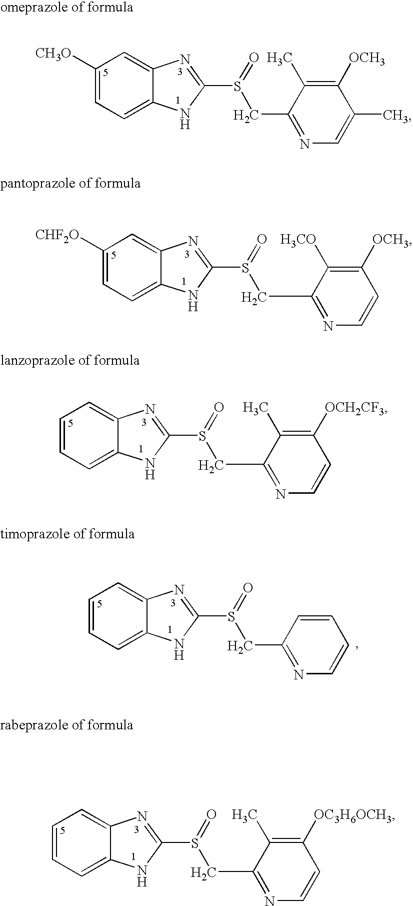
pharmaceutically acceptable salts, isomers and hydrates thereof,
and mixtures thereof,
said delivery vehicle comprising one or more members of the group consisting of pharmaceutically acceptable carriers, diluents and excipients.

2. A solid unit dosage form as defined in claim 1 wherein said gastric acid secretion inhibitor benzimidazole is selected from the group consisting of omeprazole, pharmaceutically acceptable salts, isomers and hydrates thereof and mixtures thereof.

3. A solid unit dosage form as defined in claim 2 wherein said gastric acid secretion inhibitor benzimidazole comprises esomeprazole, an isomer of omeprazole, of formula

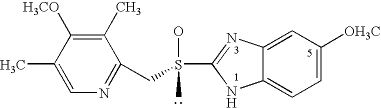

4. A solid unit dosage form as defined in claim 1 wherein said gastric acid secretion inhibitor benzimidazole is selected from the group consisting of pantoprazole, pharmaceutically acceptable salts, isomers and hydrates thereof and mixtures thereof.

5. A solid unit dosage form as defined in claim 1 wherein said gastric acid secretion inhibitor benzimidazole is selected from the group consisting of pantoprazole, and pantoprazole sodium salt.

6. A solid unit dosage form as defined in claim 4 wherein said gastric acid secretion inhibitor benzimidazole is selected from the group consisting of omeprazole and omeprazole magnesium salt.

7. A pharmaceutical solid unit dosage form for oral administration as defined in claim 1 wherein the dry prepared mixture is at least essentially free of any alkaline component.

8. A solid unit dosage form as defined in claim 7 wherein said gastric acid secretion inhibitor benzimidazole is selected from the group consisting of omeprazole, pharmaceutically acceptable salts, isomers and hydrates thereof and mixtures thereof.

9. A solid unit dosage form as defined in claim 7 wherein said gastric acid secretion inhibitor benzimidazole is selected from the group consisting of omeprazole and omeprazole magnesium salt.

10. A solid unit dosage form as defined in claim 8 wherein said gastric acid secretion inhibitor benzimidazole comprises esomeprazole, an isomer of omeprazole, of formula

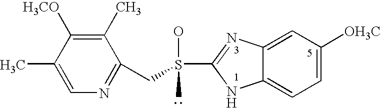

11. A solid unit dosage form as defined in claim 7 wherein said gastric acid secretion inhibitor benzimidazole is selected from the group consisting of pantoprazole, pharmaceutically acceptable salts, isomers and hydrates thereof and mixtures thereof.

12. A solid unit dosage form as defined in claim 7 wherein said gastric acid secretion inhibitor benzimidazole is selected from the group consisting of pantoprazole, and pantoprazole sodium salt.

13. A solid unit dosage form as defined in claim 7 wherein said delivery vehicle comprises a filler component, a binding agent component, a solubilizing agent component, and a surfactant component.

14. A solid unit dosage form as defined in claim 7 wherein said delivery vehicle comprises a filler component, a binding agent component, a disintegrating agent component, a solubilizing agent component, and a lubricant component and a surfactant component.

15. A solid unit dosage form as defined in claim 14 wherein said gastric acid secretion inhibitor benzimidazole is selected from the group consisting of omeprazole and omeprazole magnesium salt.

16. A solid unit dosage form as defined in claim 15 wherein the filler component is lactose, the binder component is hydroxymethylpropyl cellulose, the disintegrating agent component is selected from the group consisting of croscarmellose, sodium starch glycolate and mixtures thereof, the solubilizing agent component is polyethylene glycol, the surfactant component is sodium lauryl sulphate, and the lubricant component is sodium stearyl fumarate.

17. A pharmaceutical dosage formulation for oral administration which comprises
(a) a unit dosage core prepared by direct compression of a dry prepared mixture comprising a gastric acid secretion inhibitor benzimidazole and a delivery vehicle; and
(b) an enteric coating surrounding said unit dosage core, said enteric coating being applied directly to the unit dosage core without a separating coating between the enteric coating and said unit dosage core
said gastric acid secretion inhibitor benzimidazole being selected from the group consisting of 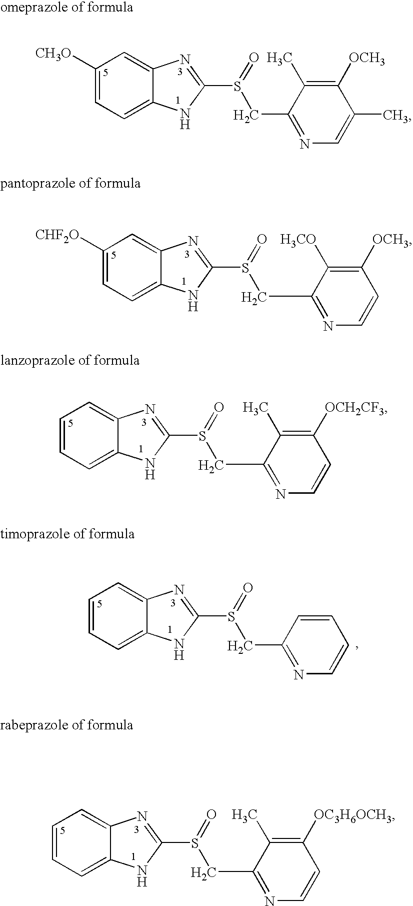
pharmaceutically acceptable salts, isomers and hydrates thereof,
and mixtures thereof,
said delivery vehicle comprising one or more members of the group consisting of pharmaceutically acceptable carriers, diluents and excipients.

18. A pharmaceutical dosage formulation as defined in claim 17 wherein said gastric acid secretion inhibitor benzimidazole is selected from the group consisting of omeprazole, pharmaceutically acceptable salts, isomers and hydrates thereof and mixtures thereof.

19. A pharmaceutical dosage formulation as defined in claim 18 wherein said gastric acid secretion inhibitor benzimidazole comprises esomeprazole, an isomer of omeprazole, of formula

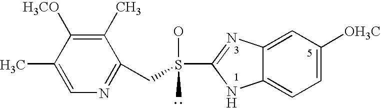

20. A pharmaceutical dosage formulation as defined in claim 17 wherein said gastric acid secretion inhibitor benzimidazole is selected from the group consisting of pantoprazole, pharmaceutically acceptable salts, isomers and hydrates thereof and mixtures thereof.

21. A pharmaceutical dosage formulation as defined in claim 17 wherein said gastric acid secretion inhibitor benzimidazole is selected from the group consisting of pantoprazole, and pantoprazole sodium salt.

22. A pharmaceutical dosage formulatio defined in claim 18 wherein said gastric acid secretion inhibitor benzimidazole is selected from the group consisting of omeprazole and omeprazole magnesium salt.

23. A pharmaceutical dosage formulation for oral administration as defined in claim 17 wherein the dry prepared mixture is at least essentially free of any alkaline component.

24. A pharmaceutical dosage formulation as defined in claim 23 wherein said gastric acid secretion inhibitor benzimidazole is selected from the group consisting of omeprazole and pharmaceutically acceptable salts, isomers and hydrates thereof and mixtures thereof.

25. A pharmaceutical dosage formulation as defined in claim 24 wherein said gastric acid secretion inhibitor benzimidazole is selected from the group consisting of omeprazole and omeprazole magnesium salt.

26. A pharmaceutical dosage formulation as defined in claim 24 wherein said gastric acid secretion inhibitor benzimidazole comprises esomeprazole, an isomer of omeprazole, of formula

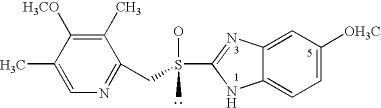

27. A pharmaceutical dosage formulation as defined in claim 23 wherein said gastric acid secretion inhibitor benzimidazole is selected from the group consisting of pantoprazole, pharmaceutically acceptable salts, isomers and hydrates thereof and mixtures thereof.

28. A pharmaceutical dosage formulation as defined in claim 27 wherein said gastric acid secretion inhibitor benzimidazole is selected from the group consisting of pantoprazole, and pantoprazole sodium salt.

29. A pharmaceutical dosage formulation as defined in claim 23, wherein the enteric coating is a methacrylic acid copolymer coating.

30. A pharmaceutical dosage formulation as defined in claim 23, wherein the enteric coating is a sugar coating

31. A pharmaceutical dosage formulation as defined in claim 23 wherein said delivery vehicle comprise a filler component, a binding agent component, a solubilizing agent component, and a surfactant component.

32. A pharmaceutical dosage formulation as defined in claim 23 wherein said delivery vehicle comprise a filler component, a binding agent component, a disintegrating agent component, a solubilizing agent component, a lubricant, and a surfactant component.

33. A pharmaceutical dosage formulation as defined in claim 32 wherein said gastric acid secretion inhibitor benzimidazole is selected from the group consisting of omeprazole and omeprazole magnesium salt.

34. A pharmaceutical dosage formulation as defined in claim 33, wherein the enteric coating is a methacrylic acid copolymer coating.

35. A pharmaceutical dosage formulation as defined in claim 33, wherein the enteric coating is a sugar coating.

36. A pharmaceutical dosage formulation as defined in claim 33 wherein the filler component is lactose, the binder component is hydroxymethylpropyl cellulose, the disintegrating agent component is selected from the group consisting of croscarmellose, sodium starch glycolate and mixtures therof, the solubilizing agent component is polyethylene glycol, the surfactant component is sodium lauryl sulphate, and the lubricant component is sodium stearyl fumarate.

37. A pharmaceutical dosage formulation as defined in claim 34 wherein the filler component is lactose, the binder component is hydroxymethylpropyl cellulose, the disintegrating agent component is selected from the group consisiting of croscarmellose, sodium starch glycolate and mixtures therof, the solubilizing agent component is polyethylene glycol, the surfactant component is sodium lauryl sulphate, and the lubricant component is sodium stearyl fumarate.

38. A pharmaceutical dosage formulation as defined in claim 35 wherein the filler component is lactose, the binder component is hydroxymethylpropyl cellulose, the disintegrating agent component is selected from the group consisiting of croscarmellose, sodium starch glycolate and mixtures therof, the solubilizing agent component is polyethylene glycol, the surfactant component is sodium lauryl sulphate, and the lubricant component is sodium stearyl fumarate.

39. A pharmaceutical dosage formulation for oral administration as defined in claim 17 wherein said dry prepared mixture comprises a gastric acid secretion inhibitor benzimidazole component, a surfactant component, a filler component, a binding agent component and a solublizing agent component; said dry prepared mixture comprising
an amount of said benzimidazole component sufficient to provide said benzimidazole component in an amount in the range of from 5 mg to 60 mg per dosage core,
an amount of said surfactant component sufficient to provide from 0.5 to 5.0 weight percent, based on the total weight of the dosage core, of said surfactant component per dosage core,
an amount of said filler component sufficient to provide from 5.0 to 85.0 weight percent based on the total weight of the core of said filler component per dosage core,
an amount of said binding agent component sufficient to provide from 1.0 to 20.0 weight percent based on the total weight of the core of said binding agent component per dosage core and
an amount of said solubilizing agent component sufficient to provide from 2.0 to 25 weight percent based on the total weight of the core of said solubilizing agent component per dosage core.

40. A pharmaceutical dosage formulation as defined in claim 39 wherein said gastric acid secretion inhibitor benzimidazole is selected from the group consisting of omeprazole and omeprazole magnesium salt.

41. A pharmaceutical dosage formulation as defined in claim 39 wherein said gastric acid secretion inhibitor benzimidazole comprises esomeprazole, an isomer of omeprazole, of formula

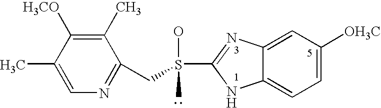

42. A pharmaceutical dosage formulation as defined in claim 39 wherein said gastric acid secretion inhibitor benzimidazole is selected from the group consisting of pantoprazole, pharmaceutically acceptable salts, isomers and hydrates thereof and mixtures thereof.

43. A pharmaceutical dosage formulation as defined in claim 42 wherein said gastric acid secretion inhibitor benzimidazole is selected from the group consisting of pantoprazole, and pantoprazole sodium salt.

44. A pharmaceutical dosage formulation as defined in claim 39 wherein the enteric coating is a methacrylic acid copolymer coating.

45. A pharmaceutical dosage formulation as defined in claim 39 wherein the enteric coating is a sugar coating

46. A pharmaceutical dosage formulation as defined in claim 39 wherein said dry prepared mixture further comprises a disintegrating agent component, and a lubricant component, said dry prepared mixture comprising
an amount of said disintegrating agent component sufficient to provide from 0.5 to 8.0 weight percent based on the total weight of the core of said disintegrating agent component per dosage core and
an amount of said lubricant agent component sufficient to provide from 0.05 to 5.0 weight percent based on the total weight of the core of said lubricant agent component per dosage core.

47. A pharmaceutical dosage formulation as defined in claim 46 wherein said gastric acid secretion inhibitor benzimidazole is selected from the group consisting of omeprazole and a magnesium salt of omeprazole.

48. A pharmaceutical dosage formulation as defined in claim 47 wherein the enteric coating is a methacrylic acid copolymer coating.

49. A pharmaceutical dosage formulation as defined in claim 47 wherein the enteric coating is a sugar coating.

50. A pharmaceutical dosage formulation as defined in claim 47 wherein the filler component comprises lactose and microcrystalline cellulose, the binder component is hydroxymethylpropyl cellulose, the disintegrating agent component is selected from the group consisiting of croscarmellose, sodium starch glycolate and mixtures therof, the solubilizing agent component is polyethylene glycol, the surfactant component is sodium lauryl sulphate, and the lubricant component is sodium stearyl fumarate.

51. A pharmaceutical dosage formulation as defined in claim 48 wherein the filler component comprises lactose and microcrystalline cellulose, the binder component is hydroxymethylpropyl cellulose, the disintegrating agent component is selected from the group consisting of croscarmellose, sodium starch glycolate and mixtures thereof, the solubilizing agent component is polyethylene glycol, the surfactant component is sodium lauryl sulphate, and the lubricant component is sodium stearyl fumarate.

52. A pharmaceutical dosage formulation as defined in claim 49 wherein the filler component comprises lactose and microcrystalline cellulose, the binder component is hydroxymethylpropyl cellulose, the disintegrating agent component is selected from the group consisting of croscarmellose, sodium starch glycolate and mixtures thereof, the solubilizing agent component is polyethylene glycol, the surfactant component is sodium lauryl sulphate, and the lubricant component is sodium stearyl fumarate.

53. A process for the manufacture of a pharmaceutical solid unit dosage form for oral administration comprising a gastric acid secretion inhibitor benzimidazole, characterized in that said process comprises a solid unit dosage form formation step wherein said dosage form is prepared by direct compression of a dry prepared mixture comprising a gastric acid secretion inhibitor benzimidazole and a delivery vehicle
said gastric acid secretion inhibitor benzimidazole being selected from the group consisting of 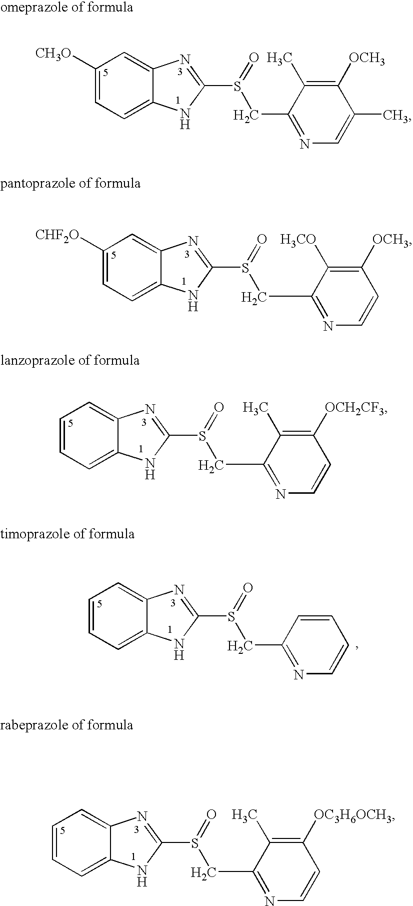
pharmaceutically acceptable salts, isomers and hydrates thereof,
and mixtures thereof,
said delivery vehicle comprising one or more members of the group consisting of pharmaceutically acceptable carriers, diluents and excipients.

54. A process as defined in claim 53 wherein the active material is selected from the group comprising omeprazole, pharmaceutically acceptable salts, isomers and hydrates thereof and mixtures thereof.

55. A process as defined in claim 54 wherein said gastric acid secretion inhibitor benzimidazole comprises esomeprazole, an isomer of omeprazole, of formula

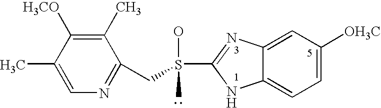

56. A process as defined in claim 53 wherein said gastric acid secretion inhibitor benzimidazole is selected from the group consisting of pantoprazole, pharmaceutically acceptable salts, isomers and hydrates thereof and mixtures thereof.

57. A process as defined in claim 53 wherein said gastric acid secretion inhibitor benzimidazole is selected from the group consisting of pantoprazole, and pantoprazole sodium salt.

58. A process as defined in claim 54 wherein said gastric acid secretion inhibitor benzimidazole is selected from the group consisting of omeprazole and omeprazole magnesium salt.

60. A process as defined in claim 53 further comprising an enteric coating application step wherein an enteric coating is applied directly on said dosage form so as to surround said dosage form without a separating layer between the enteric coating and said dosage form.

61. A process as defined in claim 60 wherein said gastric acid secretion inhibitor benzimidazole is selected from the group consisting of omeprazole, pharmaceutically acceptable salts, isomers and hydrates thereof and mixtures thereof.

62. A process as defined in claim 53 wherein the dry prepared mixture is at least essentially free of any alkaline component.

63. A process as defined in claim 62 wherein the gastric acid secretion inhibitor benzimidazole is selected from the group comprising omeprazole and pharmaceutically acceptable salts, isomers and hydrates thereof and mixtures thereof.

64. A process as defined in claim 63 wherein said gastric acid secretion inhibitor benzimidazole comprises esomeprazole, an isomer of omeprazole, of formula

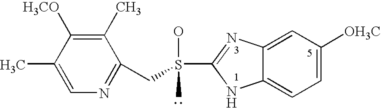

65. A process as defined in claim 62 wherein said gastric acid secretion inhibitor benzimidazole is selected from the group consisting of pantoprazole, pharmaceutically acceptable salts, isomers and hydrates thereof and mixtures thereof.

66. A process as defined in claim 65 wherein said gastric acid secretion inhibitor benzimidazole is selected from the group consisting of pantoprazole, and pantoprazole sodium salt.

67. A process as defined in claim 62 wherein the gastric acid secretion inhibitor benzimidazole is selected from the group comprising omeprazole and omeprazole magnesium salt.

68. A process as defined in claim 62 wherein said delivery vehicle comprise a filler component, a binding agent component, a solubilizing agent component and a surfactant component.

69. A process as defined in claim 68 wherein said delivery vehicle comprise a filler component, a binding agent component, a solubilizing agent component, a surfactant component, a disintegrating agent component and a lubricant.

70. A process as defined in claim 69 wherein said gastric acid secretion inhibitor benzimidazole is selected from the group consisting of omeprazole and omeprazole magnesium salt.

71. A process as defined in claim 69 wherein the filler component comprises lactose and microcrystalline, the binder component is hydroxymethylpropyl cellulose, the disintegrating agent component is selected from the group consisiting of croscarmellose, sodium starch glycolate and mixtures thereof, the solubilizing agent component is polyethylene glycol, the surfactant component is sodium lauryl sulphate, and the lubricant component is sodium stearyl fumarate.

72. A process for the manufacture of a pharmaceutical dosage formulation for oral administration comprising a gastric acid secretion inhibitor benzimidazole, characterized in that said process comprises:
(a) a solid unit dosage form formation step wherein said dosage form is prepared by direct compression of a dry prepared mixture comprising a gastric acid secretion inhibitor benzimidazole and a delivery vehicle, wherein the dry prepared mixture is at least essentially free of any alkaline component; and
(b) an enteric coating application step wherein an enteric coating is applied directly on said dosage form so as to surround said dosage form without a separating layer between the enteric coating and said dosage form
said gastric acid secretion inhibitor benzimidazole being selected from the group consisting of 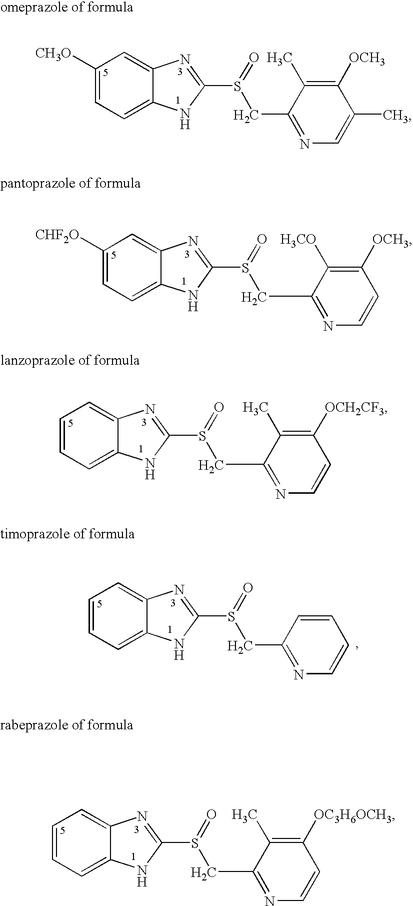
pharmaceutically acceptable salts, isomers and hydrates thereof,
and mixtures thereof,
said delivery vehicle comprising one or more members of the group consisting of pharmaceutically acceptable carriers, diluents and excipients.

73. A process as defined in claim 72 wherein said gastric acid secretion inhibitor benzimidazole is selected from the group consisting of omeprazole, pharmaceutically acceptable salts, isomers and hydrates thereof and mixtures thereof.

74. A process as defined in claim 72 wherein said gastric acid secretion inhibitor benzimidazole is selected from the group consisting of omeprazole and omeprazole magnesium salt.

75. A process as defined in claim 72, wherein the enteric coating is a methacrylic acid copolymer coating.

76. A process as defined in claim 72 wherein the enteric coating is a sugar coating

77. A process as defined in claim 72 wherein said delivery vehicle comprise one or more fillers, one or more binding agents, one or more solubilizing agents and one or more surfactants.

78. A process as defined in claim 72 wherein said delivery vehicle comprise a filler component, a binding agent component, a solubilizing agent component, a surfactant component, a disintegrating agent component and a lubricant component.

79. A process as defined in claim 78 wherein said gastric acid secretion inhibitor benzimidazole is selected from the group consisting of omeprazole and omeprazole magnesium salt.

80. A process as defined in claim 79, wherein the enteric coating is a methacrylic acid copolymer coating.

81. A process as defined in claim 79, wherein the enteric coating is a sugar coating

82. A process as defined in claim 79 wherein the filler component comprises lactose and microcrystalline, the binder component is hydroxymethylpropyl cellulose, the disintegrating agent component is selected from the group consisiting of croscarmellose, sodium starch glycolate and mixtures thereof, the solubilizing agent component is polyethylene glycol, the surfactant component is sodium lauryl sulphate, and the lubricant component is sodium stearyl fumarate.

83. A process as defined in claim 80 wherein the filler component comprises lactose and microcrystalline, the binder component is hydroxymethylpropyl cellulose, the disintegrating agent component is selected from the group consisiting of croscarmellose, sodium starch glycolate and mixtures thereof, the solubilizing agent component is polyethylene glycol, the surfactant component is sodium lauryl sulphate, and the lubricant component is sodium stearyl fumarate.

84. A process as defined in claim 81 wherein the filler component comprises lactose and microcrystalline, the binder component is hydroxymethylpropyl cellulose, the disintegrating agent component is selected from the group consisiting of croscarmellose, sodium starch glycolate and mixtures thereof, the solubilizing agent component is polyethylene glycol, the surfactant component is sodium lauryl sulphate, and the lubricant component is sodium stearyl fumarate.

85. A process as defined in claim 72 wherein said dry prepared mixture comprises a gastric acid secretion inhibitor benzimidazole component, a surfactant component, a filler component, a binding agent component and a solubilizing agent component; said dry prepared mixture comprising
an amount of said benzimidazole component sufficient to provide said benzimidazole component in an amount in the range of from 5 mg to 60 mg per dosage core,
an amount of said surfactant component sufficient to provide from 0.5 to 5.0 weight percent, based on the total weight of the dosage core, of said surfactant component per dosage core,
an amount of said filler component sufficient to provide from 5.0 to 85.0 weight percent based on the total weight of the core of said filler component per dosage core,
an amount of said binding agent component sufficient to provide from 1.0 to 20.0 weight percent based on the total weight of the core of said binding agent component per dosage core and
an amount of said solubilizing agent component sufficient to provide from 2.0 to 25 weight percent based on the total weight of the core of said solubilizing agent component per dosage core.

86. A process as defined in claim 85 wherein said gastric acid secretion inhibitor benzimidazole is selected from the group consisting of omeprazole and omeprazole magnesium salt.

87. A process as defined in claim 86 wherein said gastric acid secretion inhibitor benzimidazole comprises esomeprazole, an isomer of omeprazole, of formula

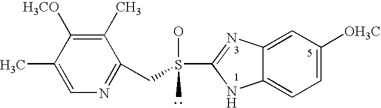

88. A process as defined in claim 85 wherein said gastric acid secretion inhibitor benzimidazole is selected from the group consisting of pantoprazole, pharmaceutically acceptable salts, isomers and hydrates thereof and mixtures thereof.

89. A process as defined in claim 88 wherein said gastric acid secretion inhibitor benzimidazole is selected from the group consisting of pantoprazole, and pantoprazole sodium salt.

90. A process as defined in claim 85 wherein the enteric coating is a methacrylic acid copolymer coating.

91. A process as defined in claim 85 wherein the enteric coating is a sugar coating

92. A process as defined in claim 85 wherein said dry prepared mixture further comprises a disintegrating agent component, and a lubricant component, said dry prepared mixture comprising
an amount of said disintegrating agent component sufficient to provide from 0.5 to 8.0 weight percent based on the total weight of the core of said disintegrating agent component per dosage core and
an amount of said lubricant agent component sufficient to provide from 0.05 to 5.0 weight percent based on the total weight of the core of said lubricant agent component per dosage core.

93. A process as defined in claim 92 wherein said gastric acid secretion inhibitor benzimidazole is selected from the group consisting of omeprazole and omeprazole magnesium salt.

94. A process stable pharmaceutical dosage formulation as defined in claim 93 wherein the enteric coating is a methacrylic acid copolymer coating.

95. A process as defined in claim 93 wherein the enteric coating is a sugar coating

96. A process as defined in claim 93 wherein the filler component comprises lactose and microcrystalline, the binder component is hydroxymethylpropyl cellulose, the disintegrating agent component is selected from the group consisting of croscarmellose, sodium starch glycolate and mixtures thereof, the solubilizing agent component is polyethylene glycol, the surfactant component is sodium lauryl sulphate, and the lubricant component is sodium stearyl fumarate.

97. A process as defined in claim 94 wherein the filler component comprises lactose and microcrystalline, the binder component is hydroxymethylpropyl cellulose, the disintegrating agent component is selected from the group consisiting of croscarmellose, sodium starch glycolate and mixtures thereof, the solubilizing agent component is polyethylene glycol, the surfactant component is sodium lauryl sulphate, and the lubricant component is sodium stearyl fumarate.

98. A process as defined in claim 95 wherein the filler component comprises lactose and microcrystalline, the binder component is hydroxymethylpropyl cellulose, the disintegrating agent component is selected from the group consisting of croscarmellose, sodium starch glycolate and mixtures thereof, the solubilizing agent component is polyethylene glycol, the surfactant component is sodium lauryl sulphate, and the lubricant component is sodium stearyl fumarate.

99. A pharmaceutical dosage formulation as defined in any one of claims 17, 22, 23, 25, 36, 37 38 and 47 wherein said enteric coating is at least essentially free of any alkaline agent component.

100. A process as defined in any one of claims 60, 72, 74, 78, 82, 83, 84, and 94 wherein said enteric coating is at least essentially free of alkaline component.
